(19) United States
(12) Patent Application Publication
(10) Pub. No.: US 2013/0084590 A1
(43) Pub. Date: Apr. 4, 2013

(54) ANALYTICAL TEST STRIP WITH BODILY FLUID PHASE-SHIFT MEASUREMENT ELECTRODES

(71) Applicant: Gustavo C. LUGO JIMENEZ, San Juan, PR (US)

(72) Inventor: Gustavo C. LUGO JIMENEZ, San Juan, PR (US)

(73) Assignee: LifeScan Scotland Ltd., Inverness-shire (GB)

(21) Appl. No.: 13/250,658

(22) Filed: Sep. 30, 2011

(51) Int. Cl. C12Q 1/54; C12M 1/34

(52) U.S. Cl. 435/14; 435/287.9

(57) ABSTRACT

An analytical test strip (“ATS”) for use with a hand-held test meter (“HHTM”) in the determination of an analyte in a bodily fluid sample (“BFS”) includes a first patterned conductive layer with a working electrode and a reference electrode, as well as a method for determining an analyte in BFS. The ATS also includes an enzymatic reagent layer disposed on the working electrode, a patterned spacer layer disposed over the first patterned conductive layer and configured to define a sample chamber (“SC”) within the ATS, and a second patterned conductive layer disposed above the first patterned conductive layer. The second patterned conductive layer includes a first phase-shift measurement electrode and a second phase-shift measurement electrode, which electrodes are disposed in the SC and are configured to measure, along with the HHTM, a phase shift of an electrical signal forced through a BFS introduced into the SC during the ATS' use.

### BACKGROUND OF THE INVENTION

1. Field of the Invention

The present invention relates, in general, to medical devices and, in particular, to analytical test strips and related methods.

2. Description of Related Art

The determination (e.g., detection and/or concentration measurement) of an analyte in a fluid sample is of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using a hand-held test meter in combination with analytical test strips (e.g., electrochemical-based analytical test strips).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified, perspective, exploded view of an analytical test strip according to an embodiment of the present invention;

FIG. 2 is a simplified top view of the analytical test strip of FIG. 1;

FIG. 3 is a simplified cross-sectional side view of the analytical test strip of FIG. 2 taken along line A-A of FIG. 2;

FIG. 4 is a simplified cross-sectional end view of the analytical test strip of FIG. 2 taken along line B-B of FIG. 2; and

FIG. 5 is a flow diagram depicting stages in a method for determining and analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, analytical test strips (e.g., an electrochemical-based analytical test strip) for use with a hand-held test meter in the determination of an analyte (such as glucose) in a bodily fluid sample (for example, a whole blood sample) include a first patterned conductive layer with at least one working electrode and a reference electrode. The analytical test strips also include an enzymatic reagent layer disposed on the working electrode, a patterned spacer layer disposed over the first patterned conductive layer and configured to define a sample chamber within the analytical test strip, and a second patterned conductive layer disposed above the first patterned conductive layer. The second patterned conductive layer includes a first phase-shift measurement electrode and a second phase-shift measurement electrode. Moreover, the first and second phase-shift measurement electrodes are disposed in the sample chamber and are configured to measure, along with the hand-held test meter, a phase shift of an electrical signal forced through a bodily fluid sample introduced into the sample chamber during use of the analytical test strip. Such phase-shift measurement electrodes are also referred to herein as bodily fluid phase-shift measurement electrodes.

Analytical test strips according to embodiments of the present invention are beneficial in that, for example, the first and second phase-shift measurement electrodes are disposed above the working and reference electrodes, thus enabling a sample chamber of advantageously low volume. This is in contrast to a configuration wherein the first and second phase-shift measurement electrodes are disposed in a co-planar relationship with the working and reference electrodes thus requiring a larger bodily fluid sample volume and sample chamber to enable the bodily fluid sample to cover the first and second phase-shift measurement electrodes as well as the working and reference electrodes.

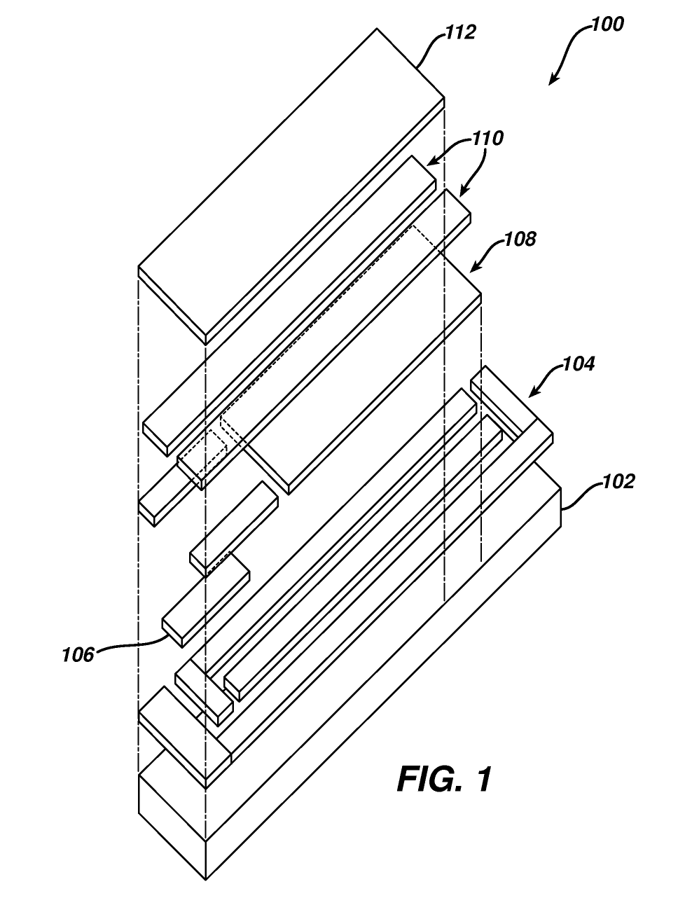

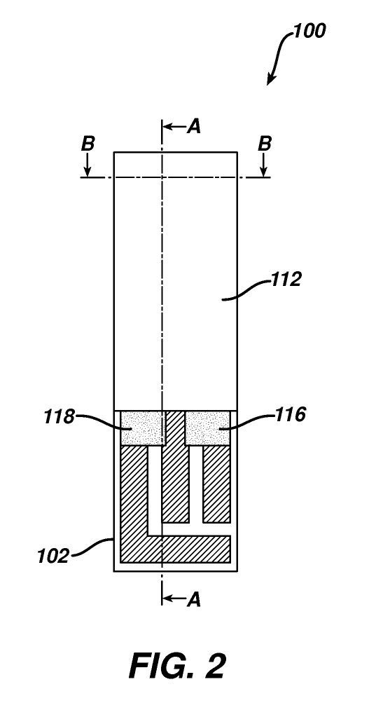

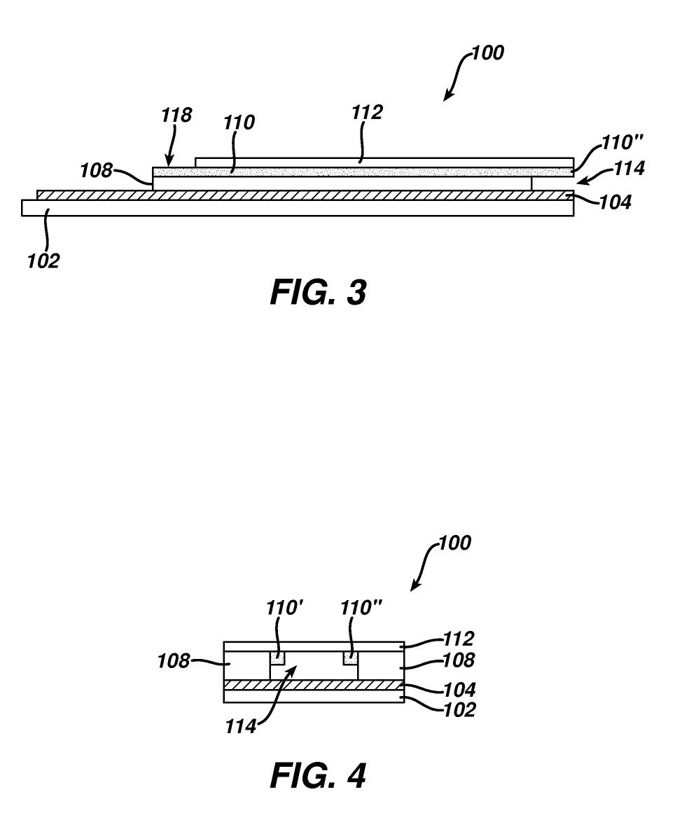

Referring to FIGS. 1 through 4, electrochemical-based analytical test strip 100 includes an electrically-insulating substrate layer 102, a first patterned conductive layer 104 disposed on the electrically-insulating substrate layer, an enzymatic reagent layer 106 (for clarity depicted in FIG. 1 only), a patterned spacer layer 108, a second patterned conductive layer 110 disposed above first patterned conductive layer 104, and an electrically-insulating top layer 112. Patterned spacer layer 108 is configured such that electrochemical-based analytical test strip 100 also includes a sample chamber 114 formed therein with patterned spacer layer 108 defining outer walls of sample chamber 114.

First patterned conductive layer 104 includes three electrodes, a counter electrode 104a (also referred to as a reference electrode), a first working electrode 104b and a second working electrode 104c (see FIG. 1).

Second patterned conductive layer 110 includes a first phase-shift measurement electrode 110′ and a second phase shift measurement electrode 110″. Second patterned conductive layer 110 also includes a first phase-shift probe contact 116 and a second phase-shift probe contact 118.

During use of electrochemical-based analytical test strip 100 to determine an analyte in a bodily fluid sample (e.g., blood glucose concentration in a whole blood sample), electrodes 104a, 104b and 104c are employed by an associated meter (not shown) to monitor an electrochemical response of the electrochemical-based analytical test strip. The electrochemical response can be, for example, an electrochemical reaction induced current of interest. The magnitude of such a current can then be correlated, taking into consideration the hematocrit of the bodily fluid sample as determined by the bodily fluid sample's phase shift, with the amount of analyte present in the bodily fluid sample under investigation. During such use, a bodily fluid sample is applied to electrochemical-based analytical test strip 100 and, thereby, received in sample chamber 114.

Electrically-insulating substrate layer 102 can be any suitable electrically-insulating substrate known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, a polystyrene substrate, a silicon substrate, ceramic substrate, glass substrate or a polyester substrate (e.g., a 7 mil thick polyester substrate). The electrically-insulating substrate can have any suitable dimensions including, for example, a width dimension of about 5 mm, a length dimension of about 27 mm and a thickness dimension of about 0.5 mm.

First patterned conductive layer 104 can be formed of any suitable electrically conductive material such as, for example, gold, palladium, carbon, silver, platinum, tin oxide, iridium, indium, or combinations thereof (e.g., indium doped tin oxide). Moreover, any suitable technique or combination of techniques can be employed to form first patterned conductive layer 104 including, for example, sputtering, evaporation, electro-less plating, screen-printing, contact printing, laser ablation or gravure printing. A typical but non-limiting thickness for the patterned conductive layer is in the range of 5 nm to 100 nm.

One skilled in the art will recognize that conventional electrochemical-based analyte test strips employ a working electrode along with an associated counter/reference electrode and enzymatic reagent layer to facilitate an electrochemical reaction with an analyte of interest and, thereby, determine the presence and/or concentration of that analyte. For example, an electrochemical-based analyte test strip for the determination of glucose concentration in a blood sample can employ an enzymatic reagent that includes the enzyme glucose oxidase and the mediator ferricyanide (which is reduced to the mediator ferrocyanide during the electrochemical reaction). Such conventional analyte test strips and enzymatic reagent layers are described in, for example, U.S. Pat. Nos. 5,708,247; 5,951,836; 6,241,862; and 6,284,125; each of which is hereby incorporated in full by reference. In this regard, the reagent layer employed in embodiments of the present invention can include any suitable sample-soluble enzymatic reagents, with the selection of enzymatic reagents being dependent on the analyte to be determined and the bodily fluid sample. For example, if glucose is to be determined in a blood sample, enzymatic reagent layer 106 can include glucose oxidase or glucose dehydrogenase along with other components necessary for functional operation.

In general, enzymatic reagent layer 106 includes at least an enzyme and a mediator. Examples of suitable mediators include, for example, ferricyanide, ferrocene, ferrocene derivatives, osmium bipyridyl complexes, and quinone derivatives. Examples of suitable enzymes include glucose oxidase, glucose dehydrogenase (GDH) using a pyrroloquinoline quinone (PQQ) co-factor, GDH using a nicotinamide adenine dinucleotide (NAD) co-factor, and GDH using a flavin adenine dinucleotide (FAD) co-factor. Enzymatic reagent layer 106 can be applied during manufacturing using any suitable technique including, for example, screen printing.

Once apprised of the present disclosure, one skilled in the art will recognize that enzymatic reagent layer 106 can, if desired, also contain suitable buffers (such as, for example, Tris HCl, Citraconate, Citrate and Phosphate), hydroxyethylcelulose [HEC], carboxymethylcellulose, ethycellulose and alginate, enzyme stabilizers and other additives as are known in the field.

Further details regarding the use of electrodes and enzymatic reagent layers for the determination of the concentrations of analytes in a bodily fluid sample, albeit in the absence of the phase-shift measurement electrodes, analytical test strips and related methods described herein, are in U.S. Pat. No. 6,733,655, which is hereby fully incorporated by reference.

Patterned spacer layer 108 can be formed of any suitable material including, for example, a 95 um thick, double-sided pressure sensitive adhesive layer, a heat activated adhesive layer, or a thermo-setting adhesive plastic layer. Patterned spacer layer 108 can have, for example, a thickness in the range of from about 1 micron to about 500 microns, preferably between about 10 microns and about 400 microns, and more preferably between about 40 microns and about 200 microns.

Second patterned conductive layer 110 can be formed of any suitable conductive material including, for example, copper, silver, palladium, gold and conductive carbon materials. Second patterned conductive layer 110 can be, for example, disposed on a lower surface of electrically-insulating top layer 112 (as depicted in FIGS. 1-4) or embedded in the lower surface of electrically-insulating top layer 112. Second patterned conductive layer 110 can have any suitable thickness including, for example, a thickness in the range of 20 microns to 100 microns.

First phase-shift measurement electrode 110′ and second phase shift measurement electrode 110″ of second patterned conductive layer 110 are separated within sample chamber 114 by a gap (in the horizontal direction of FIG. 4) that is suitable for phase-shift measurement. Such a gap can be, for example, in the range of 20 microns to 1,100 microns with a typical gap being 500 microns. Moreover, the surface area of first phase-shift measurement electrode 110′ and second phase shift measurement electrode 110″ that is exposed to a bodily fluid sample within sample chamber 114 is typically 0.5 mm² but can range, for example, from 0.1 mm² to 2.0 mm².

Electrochemical-based analytical test strip 100 can be manufactured, for example, by the sequential aligned formation of first patterned conductive layer 104, enzymatic reagent layer 106, patterned spacer layer 108, second patterned conductive layer 110 and electrically insulting top layer 112 onto electrically-insulating substrate layer 102. Any suitable techniques known to one skilled in the art can be used to accomplish such sequential aligned formation, including, for example, screen printing, photolithography, photogravure, chemical vapour deposition, sputtering, tape lamination techniques and combinations thereof.

Analytical test strops according to embodiments can be configured, for example, for operable electrical connection (via, for example, first and second phase shift probe contacts 116 and 118) and use with the analytical test strip sample cell interface of a hand-held test meter as described in co-pending patent application Ser. No. 13/250,525 [tentatively identified by attorney docket number DDI5209USNP], which is hereby incorporated in full be reference.

It has been determined that a relationship exists between the reactance of a whole blood sample and the hematocrit of that sample. Electrical modeling of a bodily fluid sample (i.e., a whole blood sample) as parallel capacitive and resistive components indicates that when an alternating current (AC) signal is forced through the bodily fluid sample, the phase shift of the AC signal will be dependent on both the frequency of the AC voltage and the hematocrit of the sample. Therefore, the hematocrit of a bodily fluid sample can be measured by, for example, driving AC signals of known frequency through the bodily fluid sample and detecting their phase shift. The phase-shift measurement electrodes of analytical test strips according to embodiments of the present invention are particularly suitable for use in such phase-shift measurements since the first and second phase shift measurement electrodes are in direct contact with a bodily fluid sample present in the sample chamber. Moreover, a bodily fluid sample hematocrit ascertained from a phase shift measurement(s) can be employed to compensate for the effect of hematocrit during analyte determination.

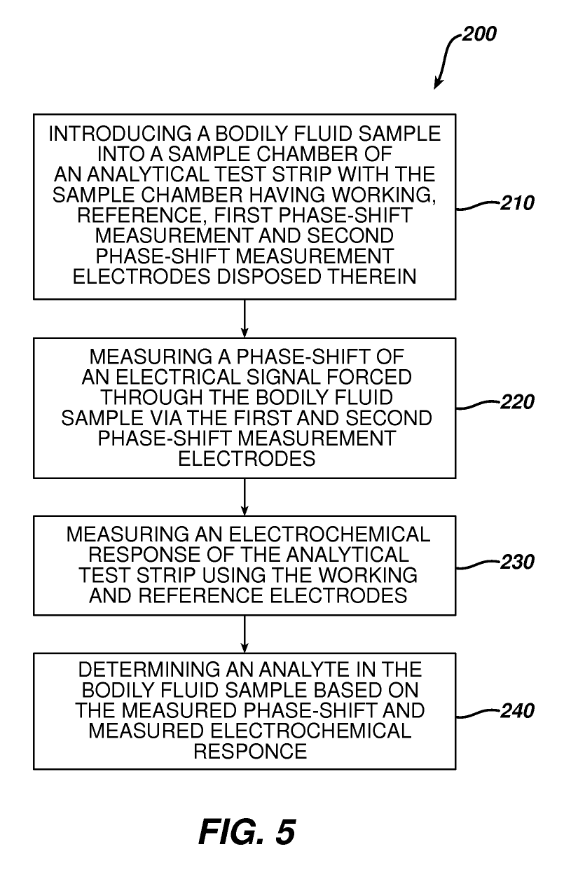

FIG. 5 is a flow diagram depicting stages in a method 200 for determining and analyte (such as glucose) in a bodily fluid sample (for example, a whole blood sample) according to an embodiment of the present invention. Method 200 includes, at step 210, introducing a bodily fluid sample into a sample chamber of an analytical test strip with the sample chamber having disposed therein a working electrode, a reference electrode, a first phase-shift measurement electrode; and a second phase-shift measurement electrode.

At step 220 of method 200, a phase shift of an electrical signal forced through the bodily fluid sample in the sample chamber via the first phase-shift measurement electrode and the second phase-shift measurement electrode is measured. In addition, method 200 includes measuring an electrochemical response of the analytical test strip using the working electrode and reference electrode (see step 230 of FIG. 5) and determining an analyte in the bodily fluid sample based on the measured phase shift and the measured electrochemical response (see step 240 of FIG. 5).

Once apprised of the present disclosure, one skilled in the art will recognize that methods according to embodiments of the present invention, including method 200, can be readily modified to incorporate any of the techniques, benefits and characteristics of analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An analytical test strip for use with a hand-held test meter in the determination of an analyte in a bodily fluid sample, the analytical test strip comprising:
a first patterned conductive layer including:
at least one working electrode; and
a reference electrode;
an enzymatic reagent layer disposed on at least the working electrode;
a patterned spacer layer disposed over the first patterned conductive layer and defining a sample chamber within the analytical test strip;
a second patterned conductive layer disposed above the first patterned conductive layer, the second patterned conductive layer including:
a first phase-shift measurement electrode; and
a second phase-shift measurement electrode, and
wherein the first phase-shift measurement electrode and the second phase-shift measurement electrode are disposed in the sample chamber and are configured to measure, along with the hand-held test meter, a phase shift of an electrical signal forced through a bodily fluid sample introduced into the sample chamber during use of the analytical test strip.

2. The analytical test strip of claim 1 wherein the first phase-shift measurement electrode and the second phase-shift measurement electrode are disposed in the sample chamber such that the first phase-shift measurement electrode and the second phase shift measurement electrode are directly exposed to bodily fluid sample introduced into the sample chamber during use of the analytical test strip.

3. The analytical test strip of claim 1further comprising:
a top electrically-insulating layer disposed above the second patterned conductive layer and having a lower surface; and
wherein the second patterned conductive layer is disposed on the lower surface of the top electrically-insulating layer.

4. The analytical test strip of claim 1further comprising:
a top electrically-insulating layer disposed above the second patterned conductive layer and having a lower surface; and
wherein the second patterned conductive layer is embedded in the lower surface of the top electrically-insulating layer.

5. The analytical test strip of claim 1 wherein the second patterned conductive layer further includes:
a first phase-shift probe contact; and
a second phase-shift probe contact.

6. The analytical test strip of claim 5 wherein the first phase-shift probe contact and the second phase shift probe contact are configured for operational electrical contact with a hand-held test meter when the analytical test strip is inserted in the hand-held test meter.

7. The analytical test strip of claim 1 wherein the first phase-shift measurement electrode and the second phase-shift measurement electrode are configured to force an electrical signal through the bodily fluid sample in the sample chamber.

8. The analytical test strip of claim 1 wherein the first phase-shift measurement electrode and the second phase-shift measurement electrode are configured to force an electrical signal of known frequency through the bodily fluid sample in the sample chamber.

9. The analytical test strip of claim 1 wherein the analytical test strip is an electrochemical-based analytical test strip configured for the determination of glucose in a whole blood sample.

10. The analytical test strip of claim 1 further comprising:
an electrically-insulating substrate layer, and wherein the first patterned conductive layer is disposed on the electrically-insulating substrate layer.

11. A method for determining an analyte in a bodily fluid sample, the method comprising:
introducing a bodily fluid sample into a sample chamber of an analytical test strip, the sample chamber having disposed therein:
at least one working electrode;
a reference electrode;
a first phase-shift measurement electrode; and
a second phase-shift measurement electrode;
measuring a phase shift of an electrical signal forced through the bodily fluid sample in the sample chamber via the first phase-shift measurement electrode and the second phase-shift measurement electrode;
measuring an electrochemical response of the analytical test strip using the at least one working electrode and reference electrode; and
determining an analyte in the bodily fluid sample based on the measured phase shift and the measured electrochemical response.

12. The method of claim 11 wherein the analyte is glucose and the bodily fluid sample is a whole blood sample.

13. The method of claim 11 wherein the first phase shift measurement electrode and the second phase shift measurement electrode are disposed above the at least one working electrode and the reference electrode.

14. The method of claim 11 wherein the bodily fluid sample is introduced into the sample chamber such that the bodily fluid sample makes direct contact with the first phase-shift measurement electrode and the second phase-shift measurement electrode.

15. The method of claim 11 wherein the first phase-shift measurement electrode and the second phase-shift measurement electrode are configured to force an electrical signal through the bodily fluid sample in the sample chamber.

16. The method of claim 11 wherein the first phase-shift measurement electrode and the second phase-shift measurement electrode are configured to force an electrical signal of a predetermined frequency through the bodily fluid sample in the sample chamber.

17. The method of claim 11 wherein the analytical test strip is an electrochemical-based analytical test strip configured for the determination of glucose in a whole blood sample.

18. The method of claim 11 wherein the first phase shift measurement electrode and the second phase shift measurement electrode are separated by a gap in the range of 20 microns to 1100 microns.

19. The method of claim 11 wherein the bodily fluid sample is introduced into the sample chamber such that the bodily fluid sample makes contact with an area of the first phase shift electrode in the range of 0.1 mm² to 2.0 mm² and makes contact with an area of the second phase shift electrode in the range of 0.1 mm² to 2.0 mm².

20. The method of claim 11 wherein the determining step employs the measured phase shift to ascertain the hematocrit of the bodily fluid sample and the ascertained hematocrit is employed in the determining of the analyte.
